(19) United States
(12) Patent Application Publication
(10) Pub. No.: US 2025/0262087 A1
(43) Pub. Date: Aug. 21, 2025

(54) CONDOM

(71) Applicant: Reckitt Benckiser Health Limited, Slough, Berkshire (GB)

(72) Inventors: Nawar AL-ZEBARI, Slough, Berkshire (GB); Anton Willem BOSMAN, Eindhoven (NL); Diana GIL-GONZALEZ, East Yorkshire (GB); Nima ROOHPOUR, Slough, Berkshire (GB)

(21) Appl. No.: 18/856,314

(22) PCT Filed: Apr. 14, 2023
(86) PCT No.: PCT/GB2023/051004

§ 371 Date: Oct. 11, 2024

(30) Foreign Application Priority Data
Apr. 14, 2022 (GB) 2205568.5

(51) Int. Cl. A61F 6/04; A61L 31/06; A61L 31/14

(52) U.S. Cl. CPC A61F 6/04; A61L 31/06; A61L 31/145; A61L 2400/16

(57) ABSTRACT

The present invention provides a condom comprising a copolymer X obtainable by polymerisation of monomers A, B, C, and D;
wherein monomer A is one or more compound of the formula:

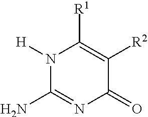

in which R¹ and R² are independently selected from H and C₁-C₂₀ alkyl optionally substituted by a hydroxyl, primary amine or secondary amine group, provided that one of R¹ and R² bears a hydroxyl, primary amine or secondary amine group;
monomer B is one or more diisocyanate compound of the formula OCN—R³—NCO, in which R³=C₄-C₂₀ alkylene, which may optionally be cyclic or branched;
monomer C is one or more telechelic hydroxy terminated polyalkylene glycol compound having a number average molecular weight of 1000 to 10,000 and is preferably linear;
monomer D is one or more diol of the formula HO—R⁴—OH, in which R⁴ is C₂-C₂₀ alkylene which may be interrupted by 1-5 heteroatoms selected from the group consisting of O, N and S, preferably 1 O atom and/or preferably wherein R⁴ is linear;
wherein, the molar ratio of monomers D to C is in the range 0.1:1 to 10:1.

### TECHNICAL FIELD

The present invention is in the field of condoms. In particular, it relates to a hydrogel condom, a packaged form of it, and a method of manufacturing it.

### BACKGROUND

Consumers today have a wide choice of different contraceptives that can be used to prevent pregnancy. Condoms, however, are the only ones which can also protect against sexually transmitted infections, such as chlamydia and HIV. A condom forms a physical barrier to the transfer of bodily fluids, viruses and other microorganisms between sexual partners. The choice of material to manufacture a condom is therefore crucial; it must have low permeability to such fluids and microorganisms, and also sufficient strength not to burst, break or tear during sexual activity.

On the other hand, a condom material should ideally be flexible and capable of stretching to accommodate different sized users. In addition, the softness of the material is a very important parameter. A significant proportion of people who choose not to use condoms do so because they do not like the feel of them. From a public health perspective as well as in the interests of the individual consumer, it would be highly beneficial to develop a material which feels more soft, natural and skin-like, whilst still being tough enough for condom use.

Most condoms on the market today are made from either natural rubber latex (“NRL”), synthetic polyisoprene (“PI”), or polyurethane (“PU”). Whist all these materials are capable of fulfilling the basic barrier function of a condom, each has its own disadvantages. NRL has an unpleasant smell and taste which needs to be masked, and causes latex allergy in a proportion of the population. Of the so-called “latex-free” materials, PU is harder and less stretchable than NRL and PI, and often feels more uncomfortable. PI still has some of the unpleasant smell and taste of NRL, and cannot be made as thin as PU currently. In addition, all these three materials have an inherent tackiness and degree of friction such that they do not feel slippery to the touch, and generally, commercial condoms made from these materials have a separate lubricant pre-applied to their surface.

A number of alternative materials have been investigated in the art. For example, carboxylated nitrile butadiene rubber has been suggested for condoms, see e.g. U.S. Pat. No. 5,195,537 (Tillotson Corporation). Other materials described in the literature include polyvinylidene chloride or low density polyethylene, mixed with polyisobutene and/or polyethylene-vinylacetate copolymer, see US 2015/0313752 (Abadi), and styrene-polyisoprene-styrene latex, see WO 2021/124215 (Church & Dwight).

WO2020/257880 (Eudaemon Technologies) describes condoms formed from a polyether-based or polyester-based polyurethane hydrogel. The polyurethane has a molecular weight between about 40,000 and 500,000 Da, and is preferably derived from polymerisation of 4,4′-dicyclohexylmethane diisocyanate with an ethyl ether or ethyl ester. WO2021/130625 (Church & Dwight) seeks to increase the inherent lubricity of a latex condom by coating it with a hydrogel/latex blend layer. The blend layer is, for example, a polyethylene oxide blended with natural rubber latex.

None of these materials have yet come to challenge the established commercial NRL, PI or PU materials on the market. Many materials which feel pleasant to the touch are inherently weak and unsuitable in the field of condoms, where minimising breakage is paramount. It is therefore a challenge to provide a condom which meets such conflicting technical requirements.

### SUMMARY OF THE INVENTION

In a first aspect of the invention there is provided a condom as set out in claim 1.

In a second aspect of the invention there is provided a package which encloses a condom according to the invention in its first aspect.

In a third aspect of the invention there is provided a method of manufacturing a condom as set out in claim 14.

In a fourth aspect of the invention there is provided the use of a film comprising a copolymer as is used in the first aspect of the invention, in hydrogel form, as a physical barrier during sexual activity.

### DETAILED DESCRIPTION

Embodiments described herein in relation to one aspect of the invention may be combined with each other and may apply equally to other aspects of the invention, unless specified to the contrary or the context otherwise requires.

The present inventors have developed a condom made from a copolymer as described herein, which is based on the copolymers outlined in WO 2006/118460, EP1972661A1 and WO2014/185779. These documents are especially interested in the provision of hydrogels which can reversibly be switched between the liquid and gelled state (for example by changing the temperature, concentration of polymer or polarity/ionic strength of the solvent). The materials are disclosed for a variety of applications, including biomedical applications such as scaffolds for tissue engineering and biodegradable implants; but use for condoms or in sexual activity is not disclosed.

Hydrogels are typically highly crosslinked polymers capable of swelling to absorb a high water content within the polymer network, upwards of 95% by weight. The water within the hydrogel offers advantageous lubrication properties. However, there needs to be a trade off between lubricity and the mechanical strength of the material. In the traditional type of hydrogel, a higher level of crosslinking results in greater material strength but lower capacity to hold water and so lower lubricity; a lower level of crosslinking may produce a more lubricious but weaker material.

In the present invention, the copolymer is reversibly crosslinked via hydrogen bonding. Monomer A reacts with monomer B to form a species that includes a ureidopyrimidone or UPy unit. Two UPy units are capable of forming a quadruple hydrogen bonding pair as a strong, reversible crosslinker, as illustrated below.

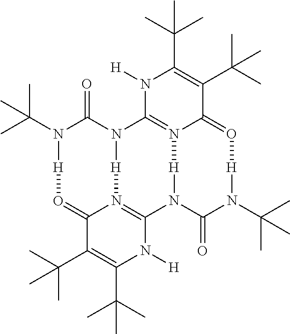

In an embodiment, monomer A has the formula

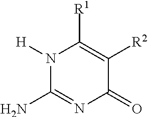

in which R¹ and R² are independently selected from H and C₁-C₂₀ alkyl optionally substituted by a single hydroxyl, primary amine or secondary amine group. Preferably, the hydroxyl or amine group is located at a terminal carbon atom. In an embodiment, one of R¹ and R² bears a single hydroxyl group, preferably on a terminal carbon. Preferably, the other of R¹ and R² is unsubstituted. In an embodiment, R² bears a hydroxyl group. In an embodiment, R¹ and R² are independently selected from H and C₁, C₂, C₃, C₄, C₅, C₆, C₇, C₈, C₉ and C₁₀ alkyl, each optionally substituted by a single hydroxyl, primary amine or secondary amine group. In an embodiment, one of R¹ and R², preferably R¹, is H or C₁-C₃ alkyl, preferably methyl or ethyl, preferably methyl. In an embodiment, one of R¹ and R² (preferably the other of the one defined in the preceding embodiment), preferably R², is C₁-C₅ alkyl (preferably C₂-C₄ alkyl, preferably C₂ or C₃ alkyl, preferably C₂ alkyl) substituted by a hydroxyl group, preferably on the terminal carbon atom, preferably —CH₂CH₂OH. In an embodiment, R¹ is H or C₁-C₃ alkyl and R² is C₁-C₅ alkyl substituted by a (preferably terminal) hydroxyl or primary amine group, preferably a hydroxyl group.

Preferably, monomer A has the formula

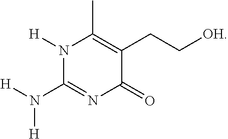

Monomer B is a diisocyanate of the formula OCN—R³—NCO, in which R³ is C₄-C₂₀ alkylene. R³ may be linear or optionally be cyclic or branched. In an embodiment, monomer B is a diisocyanate of the formula OCN—R³—NCO in which R³ is an alkylene group having: at least 5 C atoms, at least 6 C atoms, at least 7 C atoms, at least 8 C atoms, at least 9 C atoms, at least 10 C atoms, at least 11 C atoms, at least 12 C atoms, or at least 13 C atoms; and/or up to 19 C atoms, up to 18 C atoms, up to 17 C atoms, up to 16 C atoms, up to 15 C atoms, up to 14 C atoms, or up to 13 C atoms. In an embodiment, R³ is C₅-C₁₈ alkylene, preferably C₆-C₁₅ alkylene. In an embodiment, R³ is C₉-C₁₄ alkylene or C₁₀-C₁₃ alkylene. For example, R³ may be C₆ alkylene, C₉ alkylene, C₁₀ alkylene or C₁₃ alkylene. In an embodiment, R³ is C₅-C₈ linear alkylene. In an embodiment, monomer B is hexamethylene diisocyanate (HDI).

In an embodiment, R³ comprises a cyclic alkylene, preferably cyclohexylene. In an embodiment, R³ comprises 1 or 2 cyclohexylene groups. Preferably, R³ is a methylene-dicyclohexylene group. Preferably, monomer B is methylene-dicyclohexyl-4,4′-diisocyanate (HMDI). Alternatively, when R³ comprises 1 cyclohexylene group, preferably R³ is a C₉ or C₁₀ group. Preferably, monomer B is isophoronediisocyanate (IPDI).

Monomer C is a telechelic hydroxy terminated polyalkylene glycol having a number average molecular weight of 1000 to 10,000 Da. In an embodiment, the telechelic hydroxy terminated polyalkylene glycol monomer C is linear. In an embodiment, it is a polyethylene glycol, polypropylene glycol, or polyethylene-polypropylene glycol (which may, for example, be a random copolymer, alternating copolymer or block copolymer). In an embodiment, it has a number average molecular weight as determined from its hydroxyl value of: at least 1500, at least 2000, at least 2500, at least 3000, at least 3500, or at least 4000 Da; and/or no more than 8000, no more than 7000, no more than 6000, no more than 5000, or no more than 4500 Da. For example, it may have a number average molecular weight of 2000-7000, or 2500-4500 Da. In an embodiment, monomer C has a number average molecular weight of 2500-3500, preferably 2800-3200, preferably 3000 Da or about 3000 Da. In an embodiment, it has a number average molecular weight of 3500-4500, preferably 3800-4200, preferably 4000 Da or about 4000 Da. In an embodiment, monomer C has a number average molecular weight of 3000-4000, preferably 3200-3800, preferably 3400-3600, preferably 3500 Da or about 3500 Da.

Monomer D is a diol of the formula HO—R⁴—OH, in which R⁴ is C₂-C₂₀ alkylene which may be interrupted by 1-5 heteroatoms selected from the group consisting of O, N and S. R⁴ may be linear or optionally be branched, cyclic or comprise a cyclic group. In an embodiment, R⁴ is linear. In an embodiment, R⁴ is alkylene having: at least 4 C atoms, at least 5 C atoms, at least 6 C atoms, at least 7 C atoms, at least 8 C atoms, at least 9 C atoms, at least 10 C atoms, at least 11 C atoms, or at least 12 C atoms; and/or up to 18 C atoms, up to 17 C atoms, up to 16 C atoms, up to 15 C atoms, up to 14 C atoms, up to 13 C atoms, up to 12 C atoms, up to 11 C atoms, up to 10 C atoms, up to 9 C atoms, or up to 8 C atoms; in all cases optionally interrupted by 1-5 heteroatoms (preferably 1 or 2 heteroatoms) selected from the group consisting of O, N and S, preferably O. For example, R⁴ may be alkylene having 4-16 C atoms, 5-15 C atoms, 6-12 C atoms, 6-8 C atoms, or 10-14 C atoms. Preferably, R⁴ is uninterrupted by any heteroatoms. Monomer D may be, for example, 1,6-hexanediol, 1,10-decanediol or 1,12-dodecanediol. In an embodiment, R⁴ comprises a cyclic alkylene, preferably a cyclohexylene, preferably in which R⁴ is uninterrupted by any heteroatoms. Preferably, R⁴ is a dimethylene-cyclohexyl group. Preferably, monomer D is 1,4-cyclohexanedimethanol (preferably cis-trans racemic mixture). Without wishing to be bound by theory, it is believed that the cyclohexylene group may increase the toughness of the resulting polymer.

In another embodiment, R⁴ is interrupted by 1 or 2 O atoms, preferably 10 atoms. For example. R⁴ may be alkylene having 2-8 C atoms, 3-7 C atoms or 4-6 C atoms, in each case interrupted by 1 or 2 O atoms. Thus, monomer D may be, for example, diethylene glycol or triethylene glycol.

In an embodiment, monomer D is a solid at 25° C. Preferably, monomer D has a melting point of: ≥30° C., ≥40° C., ≥50° C., ≥60° C., ≥70° C., ≥75° C., or ≥80° C.; and/or ≤120° C., ≤110° C., ≤100° C., ≤90° C., or ≤85° C. For example, monomer D may have a melting point of 40-100° C., 50-90° C., or 70-85° C., or 75-83° C.

Without wishing to be bound by theory, a relatively long chain R⁴ group within monomer D, and/or relatively high melting point, may contribute towards forming a copolymer which has excellent stability towards heat degradation in storage. The resulting material may also result in condoms that are especially pleasant to the touch and/or less tackiness upon donning.

In an embodiment, the molar ratio of monomers D to C is: at least 0.5:1, at least 1:1, at least 1.5:1, at least 2:1, at least 3:1, at least 4:1, or at least 5:1; and/or no more than 9:1, no more than 8:1, no more than 7:1, no more than 6:1, no more than 5:1, no more than 4.7:1, or no more than 4.5:1. In an embodiment, the molar ratio of monomers D to C is in the range 3:1-6:1, 3.2:1-5:1, or 3.5:1 to 4.7:1.

The optimum ratio of monomers D to C may depend on the number average molecular weight of monomer C and/or the choice of monomer D. In an embodiment, monomer C has a number average molecular weight of 2500 to 3500 Da and the molar ratio of monomers D to C is 1.5-6.5, preferably monomer C has a number average molecular weight of 3000 Da and a molar ratio of monomers D to C of 2-6, 3-5, or 4. In an embodiment, monomer C has a number average molecular weight of 3500 to 4500 Da and the molar ratio of monomers D to C is 4.5-8.5, 5-8, or 5.5-7; preferably monomer C has a number average molecular weight of 4000 Da and a molar ratio of monomers D to C of 4-8, 5-7, or 6. Preferably, in the embodiments of this paragraph, monomer D is 1,6-hexanediol.

In an embodiment:
a) monomer C has a number average molecular weight of 2500 to 5000 Da, 2700 to 4500 Da, 2800 to 4200 Da, 3000 to 4000 Da, 3000 Da or about 3000 Da;
b) the molar ratio of monomers D to C is: at least 3, at least 3.2, at least 3.5, at least 4, at least 4.5, or at least 4.7; and/or up to 6, up to 5.5, up to 5.0, or up to 4.8; preferably 3-6, 3.2-5, 3.5-4.8, 4-4.7, 4.5, or about 4.5; and
c) monomer D: is a diol of the formula HO—R⁴—OH in which R⁴ is alkylene having 10-14 C atoms, preferably 11-13 C atoms, preferably 12 C atoms, preferably wherein monomer D is 1,12-dodecanediol; or has a melting point of 2 70° C., preferably 75-85° C.

In an embodiment, the molar ratio of monomer B to the sum of monomers A+C+D is: at least 0.9, at least 0.95, or at least 0.98; and/or up to 1.1, up to 1.05 or up to 1.02. Preferably, the molar ratio of monomer B to the sum of monomers A+C+D is 0.9-1.1, 0.95-1.05, 0.98-1.02, about 1, or 1.00.

In an embodiment, the copolymer X has a weight average molecular weight of 5 to 140 kDa, preferably: ≥10 kDa, ≥20 kDa, ≥30 kDa, ≥40 kDa, ≥50 kDa, ≥60 kDa, or ≥70 kDa; and/or ≤130 kDa, ≤120 kDa, ≤110 kDa, ≤100 kDa, ≤95 kDa, ≤90 kDa, ≤85 kDa, or ≤80 kDa; preferably 50-80 kDa; as determined with size-exclusion chromatography in DMF comprising 10 mM LiBr at 50° C. using PEO/PEG-standards. However, the weight average molecular weight of copolymer X will be influenced by the molecular weight of the monomers used to form it. Preferably, the weight average molecular weights described in this paragraph apply to embodiments in which monomer D is 1,6-hexanediol.

In an embodiment:
a) monomer D: is a diol of the formula HO—R⁴—OH in which R⁴ is alkylene having 10-14 C atoms, preferably 11-13 C atoms, preferably 12 C atoms, preferably wherein monomer D is 1,12-dodecanediol; or has a melting point of 2 70° C., preferably 75-85° C.; and
b) the copolymer X has a weight average molecular weight of: ≥70 kDa, ≥75 kDa, ≥80 kDa, ≥85 kDa, ≥90 kDa, ≥95 kDa, ≥100 kDa, or ≥105 kDa; and/or ≤130 kDa, ≤120 kDa, or ≤110 kDa; preferably 80-120 kDa, 85-115 kDa or 90-110 kDa.

In embodiment, the copolymer X is a random copolymer.

In an embodiment, the copolymer X is obtainable by reaction of monomers A (or a tautomer thereof), B and C together in the same reaction mixture to form a reaction product, preferably followed by polymerisation of the reaction product with monomer D.

In another embodiment, the copolymer X is obtainable by reaction of monomers A (or a tautomer thereof), B, C and D together in the same reaction mixture to form a reaction product.

The reaction may optionally be catalysed, such as with a catalyst known in the art for the reaction of isocyanate with hydroxyl groups. Examples of suitable catalysts are given in WO2006/118460 and WO2014/185779 and include tin compounds like dibutyl tin dilaurate. Alternatively, the reaction may proceed with no tin catalyst or no catalyst at all.

In an especially preferred embodiment, 2-amino-4-hydroxy-5-(2-hydroxyethyl)-6-methyl-pyrimidine (or a tautomer thereof) is reacted with a polyethylene glycol, a diisocyanate and a diol (HO—R—OH). The diisocyanate is preferably HMDI, methylenedicyclohexyl-4,4′-diisocyanate. The diol is preferably 1,6-hexanediol, 1,7-heptanediol, 1,8-octanediol, 1,4-cyclohexanedimethanol, 1,9-nonanediol, 1,10-decanediol, 1,11-undecanediol, or 1,12-dodecanediol, preferably 1,6-hexanediol, 1,4-cyclohexanedimethanol or 1,12-dodecanediol. The polyethylene glycol is preferably PEG1000-PEG6000, preferably PEG3000-PEG4000.

In an embodiment, monomer A is 2-amino-4-hydroxy-5-(2-hydroxyethyl)-6-methyl-pyrimidine, monomer B is methylenedicyclohexyl-4,4′-diisocyanate, monomer C is a linear polyethylene glycol having a number average molecular weight of 2000-6000, monomer D is 1,6-hexanediol, 1,7-heptanediol, 1,8-octanediol, 1,4-cyclohexanedimethanol, 1,9-nonanediol, 1,10-decanediol, 1,11-undecanediol, or 1,12-dodecanediol, and the molar ratio of monomers D to C is 3:1 to 8:1.

In an embodiment, monomer A is 2-amino-4-hydroxy-5-(2-hydroxyethyl)-6-methyl-pyrimidine, monomer B is methylenedicyclohexyl-4,4′-diisocyanate, monomer C is PEG2800-4500 (preferably PEG3000-4200, preferably PEG4000), monomer D is 1,6-hexanediol, and the molar ratio of monomers D to C is 3-7 (preferably 4-6.5 or 5-6). In an embodiment, monomer A is 2-amino-4-hydroxy-5-(2-hydroxyethyl)-6-methyl-pyrimidine, monomer B is methylenedicyclohexyl-4,4′-diisocyanate, monomer C is PEG2800-3500 (preferably PEG2900-3200, preferably PEG3000), monomer D is 1,12-dodecanediol, and the molar ratio of monomers D to C is 2-6 (preferably 3-5, 3.5-4.7, or 4-4.5).

In each case described above, the invention may use a mixture of different monomers A and/or mixture of different monomers B and/or mixture of different monomers C and/or mixture of different monomers D. Preferably, however, only a single monomer A and/or B and/or C and/or D is used (preferably a single of each of monomers A, B, C, and D). Where a mixture of different monomers A, B, C, and/or D is used, the quantities referenced above relate to the total quantity of the respective monomer type, for example molar ratio of D:C means molar ratio of total monomers D:molar ratio of total monomers C.

In an embodiment, the condom comprises more than one type of copolymer X. For example, different layers of the condom may comprise different copolymer X. In an embodiment, different copolymer X species are present together as a blend. For example, the blend may comprise a first copolymer X and a second copolymer X in a weight ratio of: at least 1:1, at least 1.5:1, at least 2:1, at least 3:1, at least 4:1, at least 5:1, at least 6:1, at least 7:1, at least 8:1, or at least 9:1; and/or up to 50:1, up to 40:1, up to 30:1, up to 20:1, up to 15:1, up to 10:1, or up to 9:1. In an embodiment, the blend comprises a first copolymer X and a second copolymer X in a weight ratio of 1:1 to 50:1, 1.5:1 to 20:1 or 2:1 to 9:1. In an embodiment, the first copolymer X has a higher tensile strength but higher E100 than the second polymer X. Thus, blending a softer material with a stronger material may achieve a good balance of properties.

In an embodiment, the condom is hydrated with water and the one or more copolymer X is in a hydrogel form. Preferably, the condom comprises: ≥20 wt % water, ≥30 wt % water, ≥40 wt % water, ≥50 wt % water ≥60 wt % water, ≥65 wt % water, or ≥70 wt % water; and/or ≤95 wt % water, ≤90 wt % water, ≤85 wt % water, ≤80 wt % water, or ≤75 wt % water, by total weight of the condom. For example, it may comprise 60-95 wt % water, 65-85 wt % water or 70-80 wt % water. If the water content is too high, the condom may be too weak, but a higher water content than the water content of skin is desirable for a soft and lubricious feel. In an embodiment, the condom has an equilibrium water content after 24 hours' swelling in water at 25° C. of the values stated in this paragraph. It will be appreciated that the actual water content of the condom may vary with time, depending on the environmental conditions under which it is stored.

The condom may take the typical shape of a tubular body with an open end and a closed end. It may be a “male” condom (intended to be donned onto the penis) or an “internal” condom (intended to be inserted into the vagina or anus prior to sexual activity). Preferably, it is a male condom. The condom may be overall straight walled or shaped e.g. having a variable width along its length. It may have a teat or reservoir at the closed end to hold semen.

In embodiments of the invention, the condom is: at least 120 mm long, or at least 130 mm long, at least 140 mm long, at least 150 mm long, at least 160 mm long, at least 170 mm long, or at least 180 mm long; and/or no more than 230 mm long, or no more than 220 mm long, or no more than 210 mm long, or no more than 200 mm long. In an embodiment, the condom is 120-230 mm long, or 160-200 mm long. These lengths are measured as the longest dimension from the open end to the closed end of the condom.

Optionally, the condom may have a maximum width (measured when laid flat) of: at least 40 mm, at least 45 mm, at least 50 mm, at least 51 mm, at least 52 mm, at least 53 mm, at least 54 mm, at least 55 mm, at least 56 mm, at least 57 mm, at least 58 mm, at least 59 mm, or at least 60 mm; and/or no more than 80 mm, no more than 75 mm, no more than 70 mm, no more than 65 mm, no more than 64 mm, no more than 63 mm, no more than 62 mm, no more than 61 mm, or no more than 60 mm. In an embodiment, the condom has a maximum width of 40-80 mm, or 45-70 mm. “Maximum width” means the width at the widest point of the condom (considering that the width may vary in the case of a shaped condom).

In the past, consumers have demanded thinner and thinner condoms due to the belief this will lead to a more natural sensation during sexual activity. Thinner condoms made of traditional materials are also more likely to be more transparent. However, the condoms of the present invention may offer an inherently pleasant tactile sensation and have a high transparency, such that their thickness becomes less detrimental. In an embodiment, the condom has a thickness of: at least 20 microns, at least 30 microns, at least 40 microns, at least 50 microns, at least 60 microns, at least 70 microns, at least 80 microns, at least 90 microns, or at least 100 microns; and/or up to 300 microns, up to 250 microns, up to 200 microns, up to 150 microns, or up to 120 microns. For example, it may have a thickness of 20-300 microns, 70-200 microns, 80-150 microns, or 90-120 microns.

In embodiments of the invention, the condom has a pore size less than 30 nm, less than 29 nm, less than 28 nm, less than 27 nm, less than 26 nm, or less than 25 nm.

In the process of rolling a condom, the material may be stretched. Since condoms may be capable of being stretched whilst being donned, and a rolling process may also stretch the material slightly, the dimensions stated herein are as measured in an unrolled, relaxed state of the condom. In addition, since the absorption of water causes the hydrogel material to swell, the dimensions stated herein are as measured on the condom with its equilibrium water content at 25° C. and with the condom under no load. The condom may consist only of the one or more copolymer X and water. In an embodiment, the condom may be transparent and/or colourless. Since no chemical crosslinkers, curing agents or other additives are needed to form the condom from an aqueous formulation of the copolymer(s), the condom of the invention can be made with a high degree of transparency compared to natural rubber latex condoms.

Preferably, the condom has an absorbance at 350 nm, per mm thickness of the condom, of ≤1 mm⁻¹, ≤0.9 mm⁻¹, ≤0.8 mm⁻¹, ≤0.7 mm⁻¹, ≤0.6 mm⁻¹, ≤0.4 mm⁻¹, ≤0.3 mm⁻¹, ≤0.2 mm⁻¹, ≤0.1 mm⁻¹, or ≤0.05 mm⁻¹, and/or an absorbance at 400 nm, per mm thickness of the condom, of ≤1 mm⁻¹, ≤0.9 mm⁻¹, ≤0.8 mm⁻¹, ≤0.7 mm⁻¹, ≤0.6 mm⁻¹, ≤0.4 mm⁻¹, ≤0.3 mm⁻¹, ≤0.2 mm⁻¹, ≤0.1 mm⁻¹, ≤0.05 mm⁻¹, or ≤0.02 mm⁻¹. This is measured with UV/vis spectroscopy, on a 1 cm by 1 cm film cut from the condom in an unrolled state.

On the other hand, it is possible to include desired additives in the condom to enhance its attractiveness to the consumer. For example, the condom may be coloured, e.g. by hydrating the condom in an aqueous formulation comprising one or more colourants, preferably hydrophilic colourants. Similarly, the condom may comprise any one or more of a flavourant, fragrance, sensate (such as an agent providing a warming, cooling or tingling sensation), antimicrobial, spermicide, or pH-regulator. It may comprise a pharmaceutical agent, such as an agent to prevent premature ejaculation e.g. benzocaine, or an agent to provide an orgasm enhancing effect. Alternatively or as well as being incorporated into the condom hydrating fluid, one or more of these additives may be present in the liquid from which the initial film is formed, for example the liquid that a condom former is dipped into. Another option is to include the additives in the reaction mixture for synthesis of the copolymer X, provided these additives do not interfere with the copolymerisation reaction.

If it is desired for the additive to stay in a localised area of the condom and not be spread throughout the entire condom, this can be achieved by application of a suitable formulation after the condom has been formed and hydrated. For example, a high viscosity benzocaine formulation could be dosed into the tip of the condom after manufacture.

In its hydrated state, the condom may have a high inherent lubricity. If additional lubricity is desired, however, the condom may comprise a further lubricious agent besides water, such as water-absorbing polysaccharides. The lubricious agent may be synthetic or derived from a natural resource. Examples of suitable lubricious agents include a carrageenan, alginate, agar, agarose, pectin, dextran, xanthan gum, Arabic gum, hyaluronic acid, chondroitin sulphate, starch, chitosan, cyclodextrin, guar gum, cellulose, polyacrylic acid, or polyacrylamide. It is preferred that the lubricious agent is non-gelling in the concentration used. If the lubricious agent is carrageenan, lamba carrageenan is preferred over iota carrageenan which in turn is preferred over kappa carrageenan. The lubricious agent may be introduced into the condom by hydrating it in water comprising the lubricious agent in a dissolved or dispersed state, preferably a dissolved state. Compared with traditional lubricants which are applied only to the surface of a latex condom, when a lubricious agent is introduced into a hydrogel in this manner, it becomes incorporated within the body of the condom as well as on the surface, and so is less easily sloughed off during sexual activity. Alternatively, a lubricant (preferably a hydrophilic lubricant, such as a traditional water-based lubricant) or other fluidic material could be applied to a surface of the condom after hydration. Or, a lubricious agent may be dissolved or dispersed in the liquid from which the initial film is formed, such as the dipping liquid. Another option may be to include the lubricious agent in the reaction mixture for synthesis of copolymer X.

The lubricious agent may be incorporated into the condom in any suitable amount. In an embodiment, the condom comprises the lubricious agent in an amount of: ≤5 wt %, preferably ≤4 wt %, ≤3 wt %, ≤2 wt %, ≤1 wt %, or ≤0.5 wt %; and/or ≥0.01 wt %, ≥0.02 wt %, ≥0.05 wt %, ≥0.1 wt %, ≥0.2 wt %, or ≥0.25 wt %. The desired concentration may vary according to the pH of the solution, to minimise gelling.

In its hydrated state, the condom may be capable of being rolled and unrolled easily without requiring the use of any finishing powder. Preferably, there is no finishing powder used on the condom.

In an embodiment, the condom has a Young's modulus at 100% strain (E100) of ≤5 MPa, ≤4 MPa, ≤3.5 MPa, ≤3 MPa, ≤2.5 MPa, or ≤2.0 MPa. In an embodiment, the condom has a toughness of ≥5 mJ/mm³, ≥10 mJ/mm³, ≥15 mJ/mm³, ≥20 mJ/mm³, ≥25 mJ/mm³, ≥30 mJ/mm³, ≥35 mJ/mm³, ≥40 mJ/mm³, ≥45 mi/mm³, ≥50 mJ/mm³, or ≥55 mJ/mm³. In an embodiment, the condom has a tensile strength of ≥5 MPa, ≥8 MPa, ≥10 MPa, ≥12 MPa, ≥13 MPa, ≥14 MPa, or ≥15 MPa. In an embodiment, the condom has an elongation to break of ≥400%, ≥500%, ≥600%, ≥700%, ≥800%, ≥900%, or ≥1000%.

The condom of the first aspect of the invention may be made by a process comprising dipping a former into a liquid formulation comprising copolymer(s) X by conventional techniques. Alternatively, a film comprising copolymer(s) X may be processed into a condom by thermoforming and/or vacuum forming. The condom may also be made by a process comprising extrusion or injection moulding.

When a condom is formed by dipping of a former, it may be followed by curling the edge of the material at the open end of the condom, whilst still on the former, to form a “bead”.

Preferably, after the basic condom shape has been formed, and the film dried and/or cooled down (depending on the shaping process), it is swelled with an aqueous formulation or water. In an embodiment, the condom is hydrated in water or an aqueous formulation for at least 1 minute, at least 5 minutes, at least 10 minutes, at least 30 minutes, at least 1 hour, at least 4 hours, at least 8 hours, at least 12 hours or at least 24 hours, to reach its equilibrium water content. Preferably, the hydration takes place at room temperature, or in between 20-60° C., in between 20-50° C., or in between 30-50° C. The required hydration time/temperature may vary depending on the thickness of the condom film.

The hydrated condom may then be sealed inside a package. The condom may optionally be rolled, before or after the hydration step, and prior to packaging.

The second aspect of the invention provides the condom of the first aspect, enclosed within a package. This may be any suitable package known in the art, for example a foil package or a plastic container with a lid. Preferably, the condom is provided inside the package in a rolled state. In an embodiment, there is no separate lubricant contained within the package.

In the third aspect of the invention, a condom is formed by dipping. Conventional dipping techniques may be used. For example, the former may be dipped once, or two or more times into a liquid comprising copolymer X. This liquid may be, for example, an aqueous dispersion of copolymer X, a solution of copolymer X in an organic solvent (e.g. a polar solvent such as ethanol), or copolymer X dissolved or dispersed in an organic solvent/water mix (e.g. water in ethanol, preferably 1-20% v/v, 6-15% v/v or 8-12% v/v water in ethanol). The liquid may comprise copolymer X in a concentration of: at least 1 wt %, at least 2 wt %, at least 3 wt %, at least 4 wt %, or at least 5 wt %; and/or up to 15 wt %, up to 14 wt %, up to 13 wt %, up to 12 wt %, up to 11 wt %, up to 10 wt %, or up to 9 wt %. For example, it may comprise copolymer X in a concentration of 1-15 wt %, 3-12 wt % or 5-9 wt %. The liquid may be prepared by dissolving or dispersing copolymer X into the water and/or organic solvent at elevated temperature, for example: at least 40° C., at least 50° C., at least 60° C. or at least 70° C.; and/or up to 100° C., up to 90° C., or up to 80° C. As described above, the former may be dipped sequentially into liquids comprising different copolymers X, and/or into a liquid comprising a blend of different copolymers X.

The copolymer may be dried at room temperature to form a film on the surface of the former. Alternatively, it may be dried under heating and/or reduced pressure. When the former is dipped more than once, it may be subjected to a drying step after each dip. Once the film is formed, it is hydrated in water or an aqueous formulation and the condom is removed from the former. The condom may be rolled (dried or hydrated) and a fluidic material (e.g. lubricant) may optionally be applied to the closed end of the condom whilst in its rolled state. The condom may have gone through an electrical testing process prior to rolling.

Considering that the condom material is liable to swell and expand in water, it may be necessary to use a smaller size former than the desired size of the finished condom.

The fourth aspect of the invention relates to the use of a film comprising copolymer X in hydrogel form as a physical barrier during sexual activity. The film may be in the form of a condom, as described in the first aspect of the invention. The film may also be a flat film used as a barrier during sexual activity, for example placed between the mouth and genitals or anus during oral sex. Such a product may be known in the art as a dental dam. The invention may offer enhanced sensation and pleasure, and so be more attractive to consumers, compared with existing commercialised dental dams.

### EXAMPLES

The following non-limiting examples are intended to illustrate the present invention.

### Example 1

### A) Preparation of (UPy Precursor) Monomer A

2.38 g (19 mmol) of 2-acetylbutyrolactone and 3.3 g (37 mmol) of guanidine carbonate were put to reflux in 20 ml of absolute ethanol in the presence of 5.2 ml triethylamine. The solution became yellow and turbid. After overnight heating at reflux, the solid was filtered, washed with ethanol, and suspended in water. The pH was adjusted to a value of 6-7 with an HCl solution, and the mixture was stirred. Filtration, rinsing of the residue with water and ethanol and subsequent drying of the solid gave the pure monomer A, 2-amino-4-hydroxy-5-(2-hydroxyethyl)-6-methyl-pyrimidine.

### B) Synthesis of Supramolecular Polymer Using Hexanediol

20 g (6.67 mmol) of telechelic hydroxy-terminated PEG-3000 (Mₙ of 3000) as monomer C was dried at 120° C. in vacuo for 2 hours. Subsequently, 1.13 g (6.67 mmol) of the monomer A of Example 1A, 6.99 g (26.7 mmol) of methylene dicyclohexane 4,4′-diisocyanate (HMDI) as monomer B, 50 ml dimethylformamide and one drop of dibutyltindilaurate were added. The reaction mixture was stirred at 90° C. After 1 hour, 1.56 g (13.3 mmol) of 1,6-hexanediol as monomer D was added (hexanediol:PEG molar ratio=2:1; molar ratio of monomer B:sum of monomers A+C+D=1). The reaction mixture was stirred for 8 hours at 90° C. The reaction mixture was then diluted with 50 ml of methanol and poured into 500 ml of diethylether. The precipitated polymer was dissolved into 70 ml chloroform and 70 ml methanol and poured into 500 ml diethylether. The precipitated polymer was dried in vacuo and obtained as a white solid, with a weight average molecular weight of 52 kDa.

### C) Condom Manufacture

A solution of 8-12% (v/v) water in ethanol was prepared and the polymer of Example 1B was dissolved in this at 70° C. while stirring until a homogeneous solution was obtained at a concentration of around 5-9 wt %. It was allowed to cool to 40° C. to form a solution having the required viscosity for dipping. A glass former was dipped into the polymer-containing liquid and retracted, leaving a layer of the liquid of the surface. This was allowed to dry for 1 hour at room temperature while rotating, followed by a second dip into the same liquid. This was dried at room temperature, while rotating, for 16 hours forming a second layer of hydrogel on the surface of the former. The condom and former were together submerged in water at room temperature, and after swelling for 5 minutes the hydrogel condom naturally separated from the glass. It was allowed to equilibrate in water for another 24 hours. On uptake of the water, the condom swelled and increased in diameter. The water content of the finished hydrogel was approximately 73% and the condom was approximately 240 μm thick.

In their rolled state, the condoms do not stick to themselves and can be unrolled easily onto the penis without requiring any dusting powder.

### D) Testing of Mechanical Properties

To illustrate its mechanical properties, a ring 2 cm wide was cut from the middle part of the condom. This ring was measured with a tensile tester by stretching at 50 mm/min until the material broke. Both the % elongation at break and Fₘₐₓ (maximum force measured during elongation) are dependent on the condom thickness. Since the thickness was not precisely controlled during this model condom production method, the values are not reported here. However, the following parameters are independent of condom thickness:
Stiffness—tangent of tensile curve from 0-2.5% elongation
E100—Young's modulus at 100% strain
Toughness—area under the tensile curve
Tensile strength

### Examples 2-13

The procedures of Example 1 were repeated using varying PEGs and varying molar ratios of the hexanediol. Results are summarised in the table below.

It will be seen that there is a general trend that increasing the molecular weight of the PEG monomer used, at constant hexanediol to PEG molar ratio, increases the water uptake. This is postulated to be due to an increased hydrophilic component of the copolymer. Similarly, at constant PEG molecular weight, increasing the hexanediol to PEG molar ratio decreases the water uptake. However, the effect becomes less pronounced at high PEG molecular weights. Thus, for PEG6000, the water uptake is already quite high, and it exhibits less variability with a change in hexanediol to PEG molar ratio, compared to PEG1000 which has a lower average water uptake and one that is more strongly influenced by the hexanediol to PEG molar ratio.

The Young's modulus generally shows a small increase with increasing hexanediol to PEG molar ratio, for a given PEG molecular weight, except for PEG1000 which is very strongly affected by this ratio. For tensile strength, PEG1000 seems to show a non-linear trend with hexanediol to PEG molar ratio, whereas the PEG 3000 and PEG4000 results seem to be better than PEG6000.

| | | | | Water | | | | | |
|---|---|---|---|---|---|---|---|---|---|
| Example | | Hexanediol | M_{w} | uptake | Condom | Stiffness | E100 | Toughness | Tensile strength |
| # | PEG | to PEG ratio | (kDa)¹ | (wt %)² | thickness (μm) | (MPa) | (MPa) | (mJ/mm³) | (MPa) |
| 2 | PEG1000 | 0 | 5 | 49 | 250 | 1.9 | 1.6 | 9.9 | 3.3 |
| 3 | PEG1000 | 1 | 8 | 42 | 200 | 7.1 | 4.4 | 23 | 8.6 |
| 4 | PEG1000 | 2 | 24 | 32 | 150 | 29 | — | 3.3 | 6.2 |
| 2 | PEG1000 | 0 | 5 | 49 | 250 | 1.9 | 1.6 | 9.9 | 3.3 |
| 5 | PEG3000 | 0 | 55 | 91 | 250 | 0.13 | 0.1 | 0.1 | 0.18 |
| 6 | PEG6000 | 0 | 55 | 91 | 150 | 0.17 | 0.12 | 0.4 | 0.36 |
| 4 | PEG1000 | 2 | 24 | 32 | 150 | 29 | — | 3.3 | 6.2 |
| 1 | PEG3000 | 2 | 65 | 72 | 220 | 1.7 | 1.5 | 22 | 5.6 |
| 7 | PEG6000 | 2 | 93 | 86 | 340 | 0.42 | 0.35 | 5.8 | 1.7 |
| 8 | PEG3000 | 4 | 52 | 63 | 100 | 3.0 | 3.3 | 36 | 10.4 |
| 9 | PEG6000 | 4 | 57 | 82 | 230 | 0.49 | 0.48 | 1.9 | 1.3 |
| 10 | PEG4000 | 3 | 96 | 76 | 90 | 0.80 | 1.1 | 21 | 5.9 |
| 11 | PEG4000 | 5 | 76 | 70 | 140 | 2.4 | 2.5 | 38 | 12.2 |
| 12 | PEG4000 | 6 | 61 | 64 | 140 | 3.4 | 3.3 | 42 | 14.4 |
| 13 | PEG6000 | 6 | 88 | 77 | 240 | 0.91 | 1.6 | 12 | 5.0 |

¹weight average, determined with SEC (DMF, PEO-standards)
²wt % of water in the hydrogel after swelling for 24 hours in water at room temperature

### Examples 14-22

The procedures of Example 1 were repeated analogously using varying PEGs and varying molar ratios of 1,12-dodecanediol instead of hexanediol. Results are summarised in the table below.

It will be seen that similar general trends apply when using dodecanediol. Comparing Examples 15, 18 and 21, as well as 14 vs 17, and 16 vs 19, increasing the molecular weight of the PEG monomer used, at constant dodecanediol to PEG molar ratio, increases the water uptake, and makes the condom softer and less stiff but also less tough and strong. At constant PEG molecular weight, increasing the dodecanediol to PEG molar ratio decreases the water uptake at least for PEG6000 and PEG4000. For PEG6000, the stiffness, toughness and tensile strength all increase with increasing diol:PEG molar ratio. For PEG4000, however, the toughness and tensile strength is better with a diol:PEG molar ratio of 4 than when this ratio is either 2 or 6. For PEG3000, a diol:PEG molar ratio of 4.5 produces the best toughness, softness and low stiffness of the ratios tested, whereas a ratio of 4 affords the highest tensile strength of the ratios tested.

It is clear, therefore, that it is important to select the best balance of all the required mechanical properties. Of the examples tested above, when hexanediol is used, the best results overall seem to be achieved with PEG3000 and hexanediol to PEG molar ratio of 2-4, and with PEG4000 and hexanediol to PEG molar ratio of 5-6. Examples 11 and 12 (PEG4000, hexanediol to PEG molar ratio of 5 or 6) are the most preferred of these due to the balance of high tensile strength and reasonably low E100. When dodecanediol is used, the best results overall seem to be achieved with PEG4000 and diol to PEG molar ratio of 4, and with PEG3000 and diol to PEG molar ratio of 4 to 4.5. It is postulated that a ratio up to 5 may also produce good results for PEG3000.

Of all the examples tested, Example 12 has the highest tensile strength, whilst Example 22 has the highest toughness combined with good tensile strength, adequately low stiffness and E100, and adequately high water uptake, closely followed by Example 21.

| | | | | Water | | | | | |
|---|---|---|---|---|---|---|---|---|---|
| Example | | Dodecanediol | M_{w} | uptake | Condom | Stiffness | E100 | Toughness | Tensile strength |
| # | PEG | to PEG ratio | (kDa)¹ | (wt %)² | thickness (μm) | (MPa) | (MPa) | (mJ/mm³) | (MPa) |
| 14 | PEG6000 | 2 | 87 | 87 | 100 | 0.35 | 0.2 | 2 | 0.94 |
| 15 | PEG6000 | 4 | 94 | 82 | 150 | 0.5 | 0.8 | 7 | 1.9 |
| 16 | PEG6000 | 6 | 107 | 77 | 130 | 0.8 | 0.7 | 10 | 2.4 |
| 17 | PEG4000 | 2 | 85 | 80 | 150 | 0.9 | 0.8 | 18 | 3.6 |
| 18 | PEG4000 | 4 | 117 | 73 | 110 | 1.1 | 1.2 | 32 | 7.7 |
| 19 | PEG4000 | 6 | 111 | 64 | 150 | 0.9 | 1.7 | 26 | 6 |
| 20 | PEG3000 | 3.5 | 93 | 62 | 110 | 2.6 | 2.1 | 21 | 6.2 |
| 21 | PEG3000 | 4 | 90 | 67 | 80 | 2.8 | 2.2 | 55 | 11.8 |
| 22 | PEG3000 | 4.5 | 107 | 67 | 180 | 2.1 | 1.9 | 58 | 11.2 |

¹weight average, determined with SEC (DMF, PEO-standards)
²wt % of water in the hydrogel after swelling for 24 hours in water at room temperature

The thickness of the condoms can be adjusted by adjusting the solids content of the liquid into which the former is dipped and/or by changing the number of dips.

### Porosity Testing

Condom samples were tested for leakage by filling with squid ink and visually observing any transfer of ink through the condom (at approx. 3000 Pa), following the procedure of Appl. Env. Microbiol., 1922, 3180. No transfer of ink was observed for the tested condoms.

### Transparency Testing

Examples 12 and 13 were repeated to obtain condom samples with different thickness (Example 12a-60 μm; Example 13a-400 μm). The condom samples were tested for their transparency and colour with UV/vis spectroscopy. Films were cut from the condom with typical dimensions of 1 cm by 1 cm. These cut specimens were brought onto a microscopy slide made of glass with a thickness of 1 mm. The absorbance of these glass slides with the different condom specimens were measured at 350 nm and at 400 nm with a double beam UV/vis spectrophotometer, using a microscopy slide without a condom film as a reference. The measured absorbance was divided by the thickness of the condom film to calculate the absorbance per mm thickness. Results were compared with those for two commercial condoms made from natural rubber latex and synthetic polyisoprene respectively.

The results show that, whilst for NRL and PI condoms, it is important to aim for the thinnest possible condom to achieve high transparency, with the condoms of the present invention it is possible to achieve a very high transparency even in a rather thick condom. The option to use thicker condoms may also afford different tactile effects for the consumer.

| | Film | | | | |
|---|---|---|---|---|---|
| | | | Film thickness | A350/mm | A400/mm |
| | A350 | A400 | (mm) | (mm⁻¹) | (mm⁻¹) |
| NRL condom | 0.413 | 0.315 | 0.05 | 8.26 | 6.30 |
| PI condom | 0.679 | 0.410 | 0.07 | 9.70 | 5.86 |
| Example 12a | 0.020 | 0.021 | 0.06 | 0.33 | 0.35 |
| Example 13a | 0.018 | 0.007 | 0.40 | 0.05 | 0.02 |

### Thermal Stability Testing

Rolled condoms according to the invention were put in a beaker and fully submerged in water for 24 hours at 20° C. The resulting fully swolled condom, submerged in water, was put in an oven at 70° C. for 24 hours, taking care that the condom remained fully submerged for the full period. The beaker was then removed from the oven and allowed to cool to room temperature for 4 hours. The rolled condom was then removed from the water and placed over a glass former and gently unrolled over it and observed.

The condoms made using hexanediol behaved well at room temperature, but when subjected to stability testing, there appeared to be a degree of sticking and brittleness appearing in the condoms (though this is quite extreme stability conditions and the condoms are still functional). The condoms made using dodecanediol exhibited increased thermal resistance in this respect. This is postulated to be connected to the fact that dodecanediol is more apolar and has a higher melting point (80° C. vs 40° C.). There is a stronger micro-phase separation from the hydrophilic PEG segments and the H-bonding of the UPy units become stronger due to the more apolar local environment.

1. A condom comprising a copolymer X obtainable by polymerisation of monomers A, B, C, and D;
wherein monomer A is one or more compound of the formula: 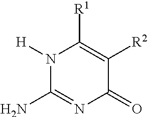
in which R¹ and R² are independently selected from H and C₁-C₂₀ alkyl optionally substituted by a hydroxyl, primary amine or secondary amine group, provided that one of R¹ and R² bears a hydroxyl, primary amine or secondary amine group;
monomer B is one or more diisocyanate compound of the formula OCN—R³—NCO, in which R³=C₄-C₂₀ alkylene, which may optionally be cyclic or branched;
monomer C is one or more telechelic hydroxy terminated polyalkylene glycol compound having a number average molecular weight of 1000 to 10,000 Da;
monomer D is one or more diol compound of the formula HO—R⁴—OH, in which R⁴ is C₂-C₂₀ alkylene which may be interrupted by 1-5 heteroatoms selected from the group consisting of O, N and S; and
wherein, the molar ratio of monomers D to C is in the range 0.1:1 to 10:1.

2. The condom as claimed in claim 1, wherein the molar ratio of monomers D to C is: at least 0.5:1, at least 1:1, at least 2:1, at least 3:1, at least 4:1, or at least 5:1; and/or no more than 9:1, no more than 8:1, no more than 7:1, or no more than 6:1.

3. The condom as claimed in claim 1, wherein monomer C is one or more compounds each having a number average molecular weight of: at least 1500 and/or no more than 9000 Da.

4. The condom as claimed in claim 1, wherein one of R¹ and R² is selected from H and unsubstituted C₁-C₂₀ alkyl, and the other of R¹ and R² is C₁-C₂₀ alkyl substituted by a hydroxyl group.

5. The condom as claimed in claim 1, wherein R³ is C₉-C₁₅ alkylene.

6. The condom as claimed in claim 1, wherein, in at least one compound of monomer D, R⁴ is C₄-C₁₈ alkylene.

7. The condom as claimed in claim 1, wherein the copolymer X is obtainable by: reaction of monomers A, B and C together in the same reaction mixture to form a reaction product, followed by polymerisation of the reaction product with monomer D; or by reaction of monomers A, B, C and D together in the same reaction mixture to form a reaction product.

8. The condom as claimed in claim 1, wherein the copolymer X is a random copolymer.

9. The condom as claimed in claim 1, wherein the copolymer X has a weight average molecular weight of 5 to 140 kDa, as determined with size-exclusion chromatography in DMF comprising 10 mM LiBr at 50° C. using PEO/PEG-standards.

10. The condom as claimed in claim 1, which has been hydrated with water such that the copolymer X is in a hydrogel form.

11. The condom as claimed in claim 10, which has been hydrated with water comprising a dissolved or dispersed lubricious agent.

12.-15. (canceled)

16. A method of manufacturing a condom, the method comprising the steps of:
providing a liquid comprising a copolymer X obtainable by polymerisation of monomers A, B, C, and D, wherein monomer A is one or more compound of the formula: 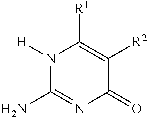
in which R¹ and R² are independently selected from H and C₁-C₂₀ alkyl optionally substituted by a hydroxyl, primary amine or secondary amine group, provided that one of R¹ and R² bears a hydroxyl, primary amine or secondary amine group;
monomer B is one or more diisocyanate compound of the formula OCN—R³—NCO, in which R³=C₄-C₂₀ alkylene, which may optionally be cyclic or branched;
monomer C is one or more telechelic hydroxy terminated polyalkylene glycol compound having a number average molecular weight of 1000 to 10,000 Da;
monomer D is one or more diol compound of the formula HO—R⁴—OH, in which R⁴ is C₂-C₂₀ alkylene which may be interrupted by 1-5 heteroatoms selected from the group consisting of O, N and S; and
wherein, the molar ratio of monomers D to C is in the range 0.1:1 to 10:1;
dipping a former into said liquid;
drying to form a film of copolymer X on the former; and
hydrating the film to form a hydrogel.

17. A method comprising placing a film against an individual during sexual activity so as to form a physical barrier, wherein the film comprises a copolymer X in hydrogel form, and wherein the copolymer X is obtainable by polymerisation of monomers A, B, C, and D, wherein monomer A is one or more compound of the formula: 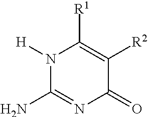
in which R¹ and R² are independently selected from H and C₁-C₂₀ alkyl optionally substituted by a hydroxyl, primary amine or secondary amine group, provided that one of R¹ and R² bears a hydroxyl, primary amine or secondary amine group;
monomer B is one or more diisocyanate compound of the formula OCN—R³—NCO, in which R³=C₄-C₂₀ alkylene, which may optionally be cyclic or branched;
monomer C is one or more telechelic hydroxy terminated polyalkylene glycol compound having a number average molecular weight of 1000 to 10,000 Da;
monomer D is one or more diol compound of the formula HO—R⁴—OH, in which R⁴ is C₂-C₂₀ alkylene which may be interrupted by 1-5 heteroatoms selected from the group consisting of O, N and S; and
wherein, the molar ratio of monomers D to C is in the range 0.1:1 to 10:1.

18. The condom as claimed in claim 1, wherein monomer C is a linear telechelic hydroxy terminated polyalkylene glycol.

19. The condom as claimed in claim 3, wherein monomer C is one or more compounds each having a number average molecular weight of 3000 to 5000 Da and a molar ratio of monomers D to C in the range 3:1 to 6:1.

20. The condom as claimed in claim 4, wherein R¹ is methyl and/or wherein R² is CH₂CH₂OH.

21. The condom as claimed in claim 5, wherein monomer B is, or the compounds of monomer B comprise, methylene-dicyclohexyl-4,4′-diisocyanate.

22. The condom as claimed in claim 6, wherein monomer D is 1,6-hexanediol, 1,4-cyclohexanedimethanol or 1,12-dodecanediol.

23. The condom as claimed in claim 9, wherein copolymer X has a weight average molecular weight of 50 to 110 kDa, as determined with size-exclusion chromatography in DMF comprising 10 mM LiBr at 50° C. using PEO/PEG-standards.

24. The condom as claimed in claim 10, wherein the condom comprises water in an amount relative to the total weight of the condom of 55-85 wt %.
